(19) United States
(12) Patent Application Publication
(10) Pub. No.: US 2001/0034322 A1
(43) Pub. Date: Oct. 25, 2001

(54) Taxol-like protein (TALP) and process for preparing the same

(75) Inventors: Byung-Doo Hwang, Taejon (KR); Kyu Lim, Taejon (KR); Sang-Tae Kwak, Chungcheongbuk-do (KR); Gi-Ryang Kweon, Daegu (KR); Wan-Hee Yoon, Taejon (KR)

Correspondence Address: DARBY & DARBY P.C., 805 Third Avenue, New York, NY, 10022, US

(73) Assignee: Byung-Doo Hwang

(21) Appl. No.: 09/799,409

(22) Filed: Mar. 5, 2001

Related U.S. Application Data
(63) Continuation of application No. 09/269,146, filed on Mar. 12, 1999.
Application No. 09/269,146 is a 371 of international application No. PCT/KR96/00158, filed on Sep. 12, 1996.

(51) Int. Cl. C12N 9/99; A61K 38/57

(52) U.S. Cl. 514/2; 435/184

(57) ABSTRACT

The present invention relates to a novel taxol-like protein (“TALP”), a process for preparing the same, and use of the same as anticancer and antiviral agents. TALP is prepared by a process which comprises the steps of homogenizing mammalian tissue, obtaining supernatant after centrifugation, and fractionating the supernatant thus obtained by cation-exchange chromatography and hydroxyapatite chromatography. TALP of the invention promotes tubulin polymerization without GTP, like taxol, and the microtubules thus polymerized are resistant to disassembly caused by Ca²⁺ and cold temperature under 4° C. and highly stabilized. Moreover, TALP of the invention inhibits cell proliferation, regeneration of scraped NIH 3T3 cell, proliferation and in vitro motility of HM7 human colon cancer cell, cell division of fertilized Xenopus oocyte, binding of tau protein to tubulin molecule, and E. coli lysis caused by virus.

### BACKGROUND OF THE INVENTION

1. Field of the Invention

The present invention relates to a novel taxol-like protein(“TALP”) and process for preparing the same, more specifically, to a novel protein which is isolated from human placental tissue and acts like taxol, a process for preparing the same, and its novel use as anticancer and antiviral agents.

2. Description of the Prior Art

It has been known that microtubule is a protein which is located mainly in cytosol and consists of heterodimeric tubulin subunits, i.e., α-tubulin and β-tubulin, and which requires GTP and microtubule-associated proteins(“MAPs”) during its assembly(see: Olmsted, J. B., 1986, Ann. Rev. Cell Biol., 2:421-457; Chen, J. et al., 1992, Nature, 360:674-677; Brandt, R. and Lee, G., 1993, J. Biol. Chem., 268:3414-3419; Maekawa, S. et al., 1992, Eur. J. Biochem., 205:195-200).

Microtubules are major component of the mitotic spindle which leads chromosomes arranged in equatorial plate to both poles during mitosis, play essential roles in many cellular functions during interphase, including maintenance of cell shape, cellular motility and attachment, and intracellular transport(see: Shiff, P. B. and Horwitz, S. B., 1981, Tubulin: A target for chemotherapeutic agents, In: Molecular Actions and Targets for Cancer Chemotherapeutic Agents., pp483-507, Academic Press, N.Y., USA; Glotz, J. S. et al., 1992, Proc. Natl. Acad. Sci., USA, 89:7026-7030; Rowinsky, E. K. et al., 1990, Nature, 82:1247-1259). They are also important in modulating the interactions of growth factors with receptors on the cell surface and the proliferative transmembrane signals generated by these interactions.

On the other hand, Vinblastine and Vincristine, etc. have anticancer activities by inhibiting microtubule assembly. On the contrary, taxol which is a kind of diterpenoid isolated from Taxus genus plant, attracts attention for the treatment of solid tumors, particularly breast cancer and uterine cancer, etc. It promotes microtubule assembly and increases stability of microtubules, which, in turn, inhibits depolymerization of microtubules, normal dynamic reorganization of microtubule network and cell proliferation, finally to show anticancer activity(see: Schiff, P. B. et al., 1979, Nature, 277:665-667; Schiff, P. B. and Horwitz, S. B., 1981, Biochemistry, 20:3247-3252; Crossin, K. L. and Carney, D. H., 1981, Cell, 27:341-350; Wilson, L. et al., 1985, Biochemistry, 24:5254-5262; Wiernik, P. H. et al., 1987, Cancer Res., 47:2486-2493). However, Taxus genus plant grows slowly in limited areas, which naturally limits the amount of taxol isolated therefrom. Moreover, environmental disruption is caused by cutting down a lot of the plants required to produce taxol.

### SUMMARY OF THE INVENTION

In accordance with the present invention, it has been discovered that a taxol-like protein(TALP) isolated from human placental tissue promotes and stabilizes microtubule assembly in vitro in a similar manner to taxol, and it can be used as an active ingredient of anticancer and antiviral agents.

A primary object of the invention is, therefore, to provide a novel taxol-like protein(TALP) which is isolated from mammalian tissues, and acts like taxol.

The other object of the invention is to provide a process for preparing TALP from mammalian tissues.

Another object of the invention is to provide a novel use of TALP as an active ingredient of anticancer and antiviral agents.

### BRIEF DESCRIPTION OF THE DRAWINGS

The above and the other objects and features of the present invention will become apparent from the following descriptions given in conjunction with the accompanying drawing, in which:

FIG. 1 is a photograph showing SDS-PAGE pattern of TALP which is isolated from human placental tissue.

FIG. 2(A) is a photograph showing SDS-PAGE pattern of fractions obtained in the course of TALP purification from human placental tissue.

FIG. 2(B) is a photograph showing corresponding Western blot analysis of SDS-PAGE as shown in FIG. 2(A).

FIG. 3 is a graph showing the effect of TALP on microtubule polymerization compared with GTP and taxol.

FIG. 4 is a graph showing the effect of TALP on microtubules assembled by taxol.

FIG. 5 is a graph showing the effect of Ca²⁺ on microtubules assembled by GTP, taxol or TALP.

FIG. 6 is a graph showing the effect of low temperature(4° C.) on microtubules assembled by GTP, taxol or TALP.

FIG. 7(A) is a photograph showing SDS-PAGE pattern of supernatants and pellets which are obtained by centrifugation of reaction mixtures for microtubule assembly in the presence of GTP, taxol and TALP, respectively.

FIG. 7(B) is a photograph showing SDS-PAGE pattern of supernatants and pellets which are obtained by centrifugation of reaction mixtures for microtubule assembly in the presence of various concentration of TALP.

FIG. 8 is a graph showing the effect of TALP on lysis of E. coli by virus.

FIG. 9 is a graph showing the results of wavelength scanning of TALP and BSA(bovine serum albumin).

FIG. 10(A) is a photograph showing SDS-PAGE pattern after coprecipitation to investigate binding of TALP with αβ-tubulin or αβ_{S}-tubulin.

FIG. 10(B) is a photograph showing SDS-PAGE pattern after coprecipitation to investigate binding of TALP with αβ-tubulin or α_{S}β_{S}-tubulin.

FIG. 11(A) is a photograph showing SDS-PAGE pattern after coprecipitation to investigate binding of tau protein and MAP2 with tubulin molecule.

FIG. 11(B) is a photograph which shows corresponding Western blot analysis of SDS-PAGE as shown in FIG. 12(A) employing monoclonal antibody against tau protein.

FIG. 11(C) is a photograph which shows corresponding Western blot analysis of SDS-PAGE as shown in FIG. 12(A) employing monoclonal antibody against MAP2.

FIG. 12(A) is an electron micrograph of tau protein immunogold labelling in the presence of GTP.

FIG. 12(B) is an electron micrograph of tau protein immunogold labelling in the presence of TALP.

FIG. 13 is a graph showing the inhibited proliferation of HM7 human colon cancer cell by TALP.

FIG. 14 is a graph showing the inhibited cellular motility of HM7 human colon cancer cell by TALP.

### DETAILED DESCRIPTION OF THE INVENTION

In order to isolate TALP from mammalian tissues such as human placenta, the tissue was homogenized and centrifuged to obtain supernatant. The supernatant thus obtained was loaded on a cation-exchange column, and the column was washed. Then, TALP containing fraction was eluted from the column, and followed by hydroxyapatite chromatography, finally to purify about 35 kDa of TALP.

It has been reported that MAPs promote tubulin polymerization in the presence of GTP, and the microtubules thus polymerized are depolymerized in the presence of Ca²⁺ at cold temperature under 4° C., while taxol promotes tubulin polymerization without GTP, and the microtubules thus polymerized are resistant to disassembly by Ca²⁺ and cold temperature under 4° C. and highly stabilized(see: Schiff, P. B. et al., 1979, Nature, 277:665-667; Schiff, P. B. and Horwitz, S. B., 1981, Biochemistry, 20:3247-3252; Crossin, K. L. and Carney, D. H., 1981, Cell, 27:341-350; Wilson, L. et al., 1985, Biochemistry, 24:5254-5262; Wiernik, P. H. et al., 1987, Cancer Res., 47:2486-2493).

In this connection, characterization of TALP purified from mammalian tissue was carried out to elucidate that TALP promotes tubulin polymerization without GTP, like taxol, and the microtubules thus polymerized are not depolymerized by Ca²⁺ and cold temperature under 4° C. and stabilized far better than taxol, which suggests that anticancer activity of TALP by the inhibition of cell proliferation is much higher than that of taxol. Also, it was revealed that microtubule assembly is caused by the direct binding of TALP on microtubule, which is also similar action mechanism of TALP to taxol.

Accordingly, ‘TALP’ of the invention refers to a protein which promotes tubulin polymerization without GTP or MAPs, the microtubules thus polymerized being resistant to Ca²⁺ and cold temperature under 4° C. and highly stabilized.

TALP is digested with trypsin to give at least 7 fragments, whose accurate number depends on factors such as specific activity of trypsin, concentration of TALP, ionic strength, pH, temperature, and incubation time.

Tau protein and MAPs such as MAP2 promote tubulin polymerization by binding to carboxyl-terminal moiety of tubulin. In the case of TALP, it binds to tubulin with tau protein in a competitive manner, not MAP2, which is also visualized by electron microscopy employing tau protein immunogold labelling. Accordingly, TALP may be applied for the development of an agent for the treatment of Alzheimer's disease, since TALP inhibits binding of tau protein to tubulin competitively.

TALP acts like taxol in vitro. Therefore, in order to examine whether TALP can be used as anticancer drug like taxol, the effects of TALP on regeneration of scraped NIH 3T3 cell, proliferation and in vitro motility of HM7 human colon cancer cell, cell division of fertilized Xenopus oocyte, were investigated. Also, effect of TALP on metastasis into liver was investigated employing injection of human colon cancer cell into spleen of nude mice. As a result, it was found that TALP inhibits cell proliferation like taxol, i.e., it inhibits regeneration of scraped NIH 3T3 cell, proliferation and in vitro motility of HM7 human colon cancer cell, cell division of fertilized Xenopus oocyte, respectively. It was also revealed that TALP inhibits markedly metastasis of HM7 into liver. Accordingly, TALP of the invention can be used as anticancer drug, anti-metastasis and anti-Alzheimer's disease drugs. And, in an experiment of E. coli lysis by virus, it was found that pretreatment of virus with TALP can not bring about E. coli lysis, which suggested that TALP of the invention can be used as antiviral agent.

The present invention is further illustrated in the following examples, which should not be taken to limit the scope of the invention. Particularly, the invention covers all proteins showing the same properties and characters of TALP of the invention regardless of the source of the protein, not limited to a 35 kDa-protein isolated from human placenta described in the following examples.

### EXAMPLE 1

### Isolation of TALP from Human Placenta

Human term placenta was washed several times with 0.154M NaCl solution until blood was completely removed. Washed placenta was cut into small pieces and 250 ml of 0.6M phosphate buffer A(0.6M potassium phosphate, 1 mM DTT, 1 mM EDTA, 0.5 mM PMSF, 0.5 μg/ml leupeptin, and 10% glycerol, pH 7.0) was added to 100 g of placenta and homogenized with Polytron(Kinemetica™, Switzerland) for 2 minutes, and stood on ice for 1 hour to obtain crude extract of TALP.

The extract thus obtained was filtered with two sheets of cheesecloth, and centrifuged at 30,000 xg for 30 minutes to obtain the supernatant. The supernatant was loaded on a phosphocellulose column equilibrated with 0.6M phosphate buffer A. The column was washed sufficiently with the same buffer, and TALP was subsequently eluted with a linear gradient from 0.6M to 1.8M phosphate. Measurement of TALP activity confirmed that TALP was eluted in a range of 1.0M to 1.3M phosphate.

Active fractions containing TALP were collected and dialyzed against glycerol solution(10% glycerol containing 1 mM DTT, 1 mM EDTA, 0.5 mM PMSF and 0.5 μg/ml leupeptin). Then, centrifugation was carried out, and the supernatant was loaded on hydroxyapatite column equilibrated with 0.35M phosphate buffer A. The column was washed with the same buffer, and TALP was subsequently eluted with a linear gradient from 0.35M to 0.55M phosphate. Measurement of TALP activity demonstrated that TALP was eluted in a range of 0.43M to 0.48M phosphate.

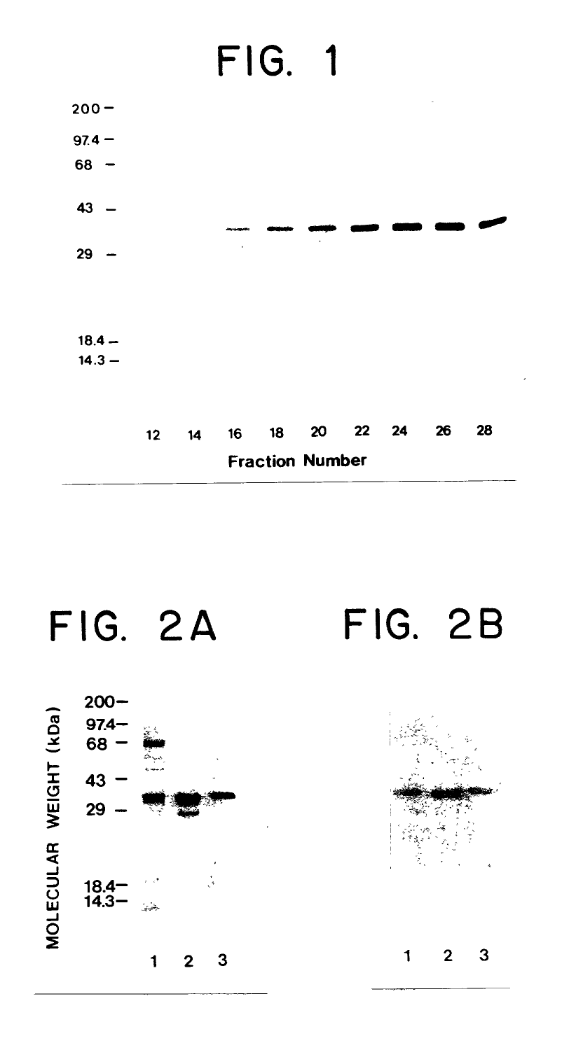

Active fractions were-collected and diluted 3 times with glycerol solution, and fractionated on hydroxyapatite column with the, same procedure as mentioned above. 12% SDS-PAGE of eluted fractions revealed that single band appeared with an apparent molecular weight of 35 kDa(see: FIG. 1).

All of the purification steps were carried out under 4° C., and TALP activity was determined by the conventional tubulin polymerization which was monitored by turbidity(see: Gaskin, F. et al., 1974, J. Mol. Biol., 89:737-758): Tubulin prepared according to the method of Hamel et al. or Asnes et al.(see: Hamel E. et al., 1981, Arch. Biochem. Biophys., 209:29-40; Asnes, C. F. and Wilson, L., 1979, Anal. Biochem., 98:64-73) was added to 0.1M MES(2-[N-morpholino]ethane sulfonic acid) buffer(0.1M MES containing 1 mM EGTA and 1 mM MgCl₂, pH 6.8) to a final concentration of 1 mg/ml, and TALP containing solution was added to a final reaction volume of 250 μl. The reaction mixture was incubated at 37° C. for 30 minutes, and the change in absorbance at 350 nm was monitored with a Gilford Model 2600 spectrophotometer(Gilford, USA).

### EXAMPLE 2

### Identification of TALP by Western Blot Analysis

In order to prepare polyclonal antibody against TALP, TALP purified in Example 1 was separated by 12% SDS-PAGE, and protein band corresponding to molecular weight of 35 kDa was excised to obtain the protein. The same volume of Freund's complete adjuvant was added to the obtained protein, mixed well, and injected separately to subcutaneous parts and leg muscles of 1.5 kg of male rabbit. Then, monthly injection of the same amount of the protein mixed with incomplete adjuvant was carried out twice(see: Harlow, E. and Lane, D., 1988, Antibodies: Laboratory Manuals. In: Immunization, pp53-137, Cold Spring Harbor Laboratory). After ten days, antibody production was confirmed by enzyme-linked immunosorbent assay(ELISA). Then, serum was obtained from the rabbit, and polyclonal antibody was purified by protein A-Sepharose affinity chromatography. The concentration of antibody thus purified was 4.0 mg/ml.

To investigate binding specificity of TALP employing polyclonal antibody thus prepared, Western blot analysis was carried out: 12% SDS-PAGE gel(11×16 cm) of 1.5 mm-width was prepared by the modification of the method of Laemmli(see: Laemmli, U., 1970, Nature, 227:680-685) Then, protein solutions, i.e., placenta homogenate, eluates of phosphocellulose chromatography and the second hydroxyapatite chromatography, each of which was obtained in Example 1, were analyzed by SDS-PAGE in a current of 20 mA per width of the gel employing slab-gel electrophoresis(Bio-Rad, USA)(see: FIG. 2(A)). Then, proteins thus separated were electro-transferred onto a nitrocellulose membrane, and immunostained with polyclonal antibody against TALP prepared above. As a result, it was found that 35 kDa-protein which exists in all the SDS-PAGE loading samples was strongly detected by the polyclonal antibody, and also, about 32 kDa-protein was weakly detected by the polyclonal antibody(see: FIG. 2(B)). In FIGS. 2(A) and 2(B), lanes 1, 2 and 3 shows placenta homogenate, eluate of phosphocellulose chromatography, and eluate of the second hydroxyapatite chromatography, respectively.

Therefore, it was determined that the prepared polyclonal antibody can be used for detection of TALP in tissues and organelles and quantification of TALP, based on the specific binding of the antibody to TALP.

### EXAMPLE 3

### Effect of TALP on Tubulin Polymerization

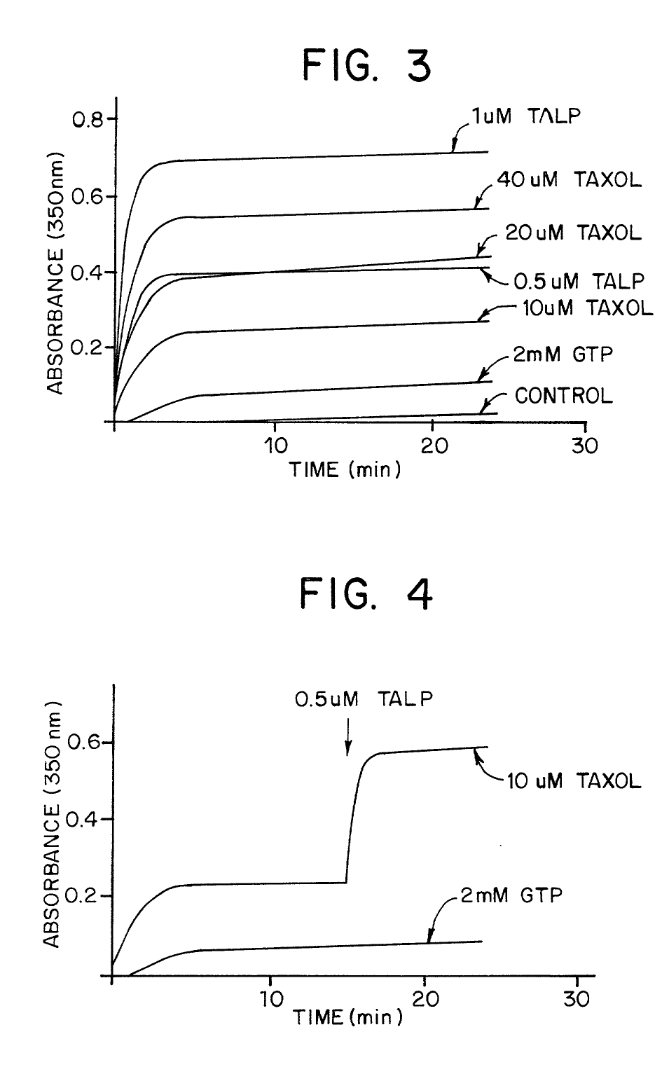

Effects of GTP, taxol and TALP on tubulin polymerization were examined in the same manner as described in Example 1. FIG. 3 demonstrates that: in the presence of 2 mM GTP(control), tubulin polymerization occurs in a type of typical sigmoid curve showing lag time of 1 to 2 minutes; in the presence of 0.5 μM TALP, tubulin polymerization occurs 2.5 times as high as the control in a type of hyperbolic curve, and in the presence of 1 μM TALP, it occurs 2.5 times as high as the control, which demonstrates that TALP promotes tubulin polymerization in a dose-dependent manner; and, in the presence of 20 μM taxol, tubulin polymerization occurs 2.5 times as high as the control whose activity is equivalent to that of 0.5 μM TALP, which demonstrates that TALP promotes tubulin polymerization about 40 times as strong as taxol. Moreover, it was revealed that TALP promotes tubulin polymerization without GTP, like taxol.

### EXAMPLE 4

### Effect of TALP on Microtubules Assembled by Taxol

To investigate effect of TALP on microtubules assembled by taxol, 10 μM of taxol was added to a reaction solution for polymerization assay as described in Example 1. When 0.5 μM of TALP was added to the reaction mixture, tubulin polymerization occurred twice or more as high as one without TALP(see: FIG. 4).

### EXAMPLE 5

### Effect of Ca²⁺ on Tubulin Polymerization

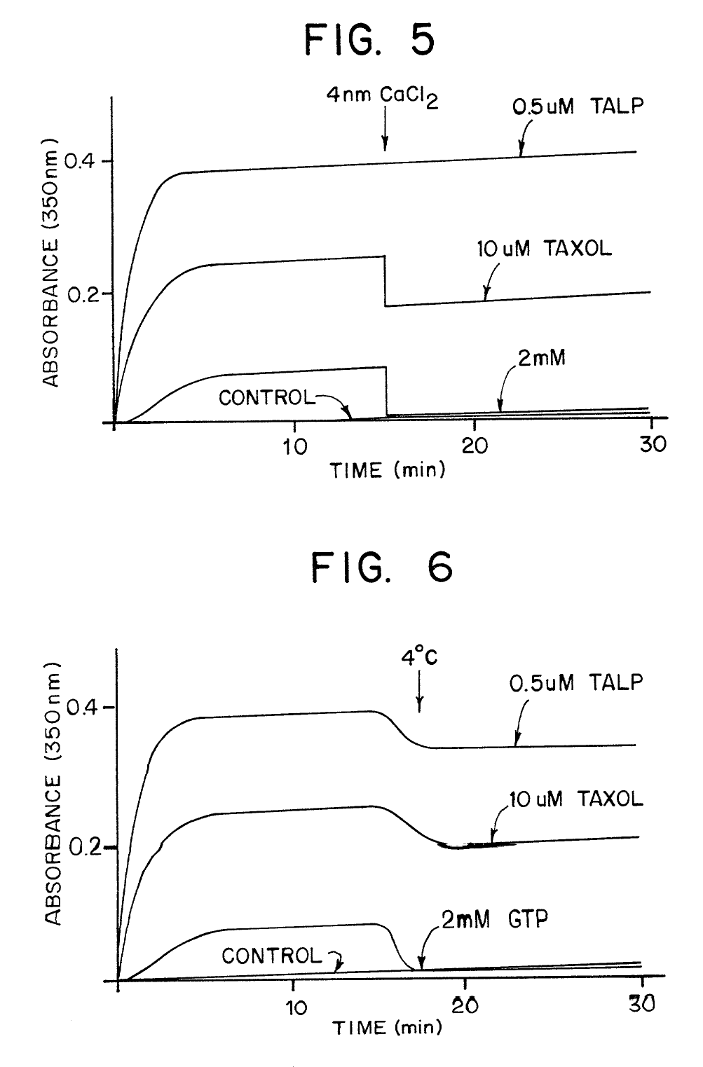

To investigate effect of Ca²⁺ on tubulin polymerization, tubulin was added to 0.1M MES buffer(pH 6.8) free of EGTA, and GTP, taxol or TALP was added, and polymerization of microtubules were carried out at 37° C. for 15 minutes. Then, 4 mM CaCl₂ was added to the reaction mixture, and depolymerization of microtubules assembled was measured, with the addition of GTP, taxol or TALP(see: FIG. 5). As shown in FIG. 5, most of microtubules assembled by 2 mM GTP were depolymerized, 30% of microtubules assembled by 10 μM taxol were also depolymerized, however none of microtubules assembled by 0.5 μM TALP were depolymerized. Accordingly, it was clearly demonstrated that TALP-stabilized microtubules were resistant to depolymerization caused by Ca²⁺.

### EXAMPLE 6

### Effect of Cold Temperature(4° C.) on Tubulin Polymerization

To investigate effect of cold temperature on tubulin polymerization, tubulin was added to 0.1M MES buffer(pH 6.8). Then GTP, taxol or TALP was added to the reaction mixture, and polymerization of microtubules were carried out at 37° C. for 15 minutes. Then, the reaction solution was chilled to a temperature of 4° C., and depolymerization of microtubules assembled was measured, with the addition of GTP, taxol or TALP(see: FIG. 6). As shown in FIG. 6, 25% and 10% of microtubules assembled by 10 μM taxol and 0.5 μM TALP, respectively, were depolymerized, while most of microtubules assembled by 2 mM CTP were depolymerized. Accordingly, it was clearly demonstrated that TALP-stabilized microtubules were resistant to depolymerization by cold temperature(4° C.) like taxol-stabilized microtubules.

### EXAMPLE 7

### Identification of Coprecipitation of Tubulin and TALP

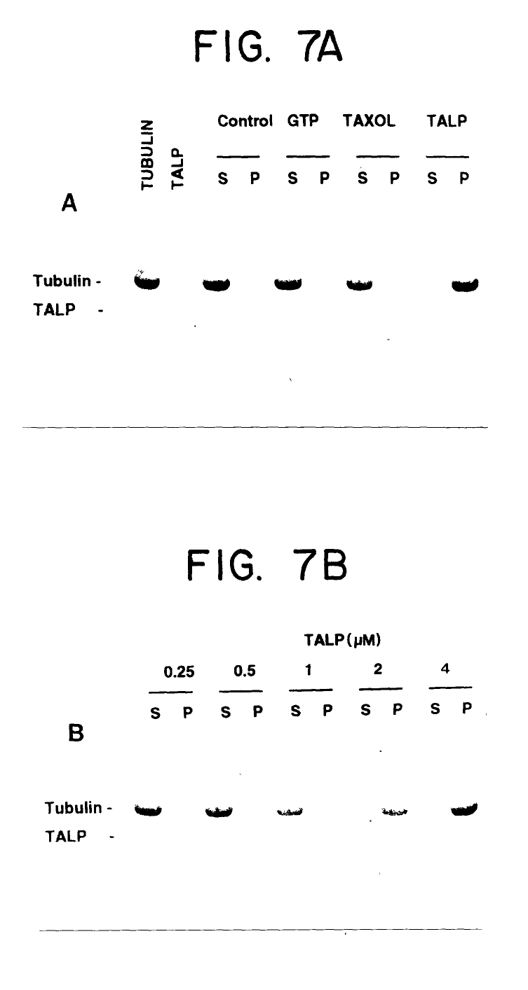

To investigate whether TALP promotes microtubule assembly in the same manner as taxol, tubulin isolated from bovine brain was added to 0.1M MES buffer(pH 6.8) to a concentration of 0.4 mg/ml. Then, 2 mM GTP, 10 μM taxol or 4 μM TALP was added to the reaction mixture, and polymerization of microtubules were carried out at 37° C. for 30 minutes. The reaction mixtures were centrifuged for 20 minutes, and the supernatants and the pellets thus obtained were analyzed by 12% SDS-PAGE(see: FIG. 7(A)). As can be seen in FIG. 7(A), about 20% and most of tubulins were polymerized in the presence of 10 μM taxol and 4 μM TALP, respectively, while most of tubulins were not polymerized in the presence of 2 mM GTP.

Also, tubulin polymerization was investigated with the variation of TALP concentration. As a result, microtubule assembly increased progressively in accordance with the increase of TALP concentration (see: FIG. 7(B)), which corresponds with the result of FIG. 3. In FIGS. 7(A) and 7(B), S and P designate supernatant and pellet, respectively. As shown in FIGS. 7(A) and 7(B), TALP did not exist in the supernatant, but was precipitated together with microtubules. Thus, it was suggested that TALP promotes microtubule assembly by direct binding of TALP to microtubule, which corresponds with the action of taxol.

### EXAMPLE 8

### Electron Microscopic Analysis of Microtubules

When tubulin polymerization was monitored by measuring the absorbance as in Example 1, increase in the absorbance may occur not only by microtubule assembly but also by the aggregation of tubulins. Thus, in order to confirm that the turbidity change is caused by microtubule assembly, the morphology of the polymers was examined with electron microscopy. Tubulin was added to 0.1M MES buffer to a concentration of 1 mg/ml, and GTP, taxol or TALP was added to a final reaction volume of 250 μl. The reaction mixture was incubated at 37° C. for 30 minutes, and the absorbance change at 350 nm was monitored. At this time, the reaction solutions showing increase in the absorbance were fixed and applied to a carbon-coated grids, and dried. Grids were then stained with 1% uranyl acetate for 5 minutes, washed with distilled water, and dried. And then, they were examined with electron microscope(Hitachi H-600, Japan) at an accelerated voltage of 75 kv. As a result, it was confirmed that typical microtubules were assembled in all reaction solutions with the addition of GTP, taxol or TALP.

Results of Examples 3 to 8 revealed that TALP of the invention, like taxol, promotes tubulin polymerization without GTP or MAPs and the microtubules thus polymerized are resistant to disassembly caused by Ca²⁺ and cold temperature under 4° C. and stabilized far better than taxol, which suggests that anticancer activity of TALP by the inhibition of cell proliferation described later is much higher than that of taxol.

### EXAMPLE 9

### Inhibition of Cell Proliferation by TALP

TALP acts like taxol in vitro. Therefore, in order to examine whether TALP can be used as anticancer agent like taxol, effects of TALP on regeneration of scraped NIH 3T3 cell and cell division of fertilized Xenopus oocyte were examined.

### EXAMPLE 9-1

### Inhibition of Regeneration of Scraped NIH 3T3 Cell

NIH 3T3 cell line(ATCC CRL 6442) was cultured in RPMI 1640 medium to a state of 95% growth. Then, cells were scraped by scratching the culture dishes with a sterilized loop, and TALP was added to the medium to observe the effect of TALP on regeneration of scraped NIH 3T3 cell. As a result, cells without TALP treatment were regenerated, but cells treated with 1 μM TALP were not regenerated. Accordingly, it was determined that TALP can be used as anticancer agent, since TALP inhibits cell proliferation like taxol.

### EXAMPLE 9-2

### Inhibition of Cell Division of Fertilized Xenopus oocyte

Microinjection of a physiological saline solution as a control or TALP into one cell of a frog fertilized egg of two stage cells was carried out employing microinjector(Drummond Scientific, USA) (see: Hiramoto, Y., 1962, Exp. Cell. Res., 27:416-426). At this time, microinjection volume was cautiously controlled in an error range of less than 0.5%. Under an assumption that half of total volume corresponds to yolk platelet in the case of 1.2 nm of fertilized egg, volume of cytosol was regarded as 450 nl, which was employed in the calculation of final concentration of injected solution.

As a result, it was determined that a control cell proliferated rapidly, but a cell injected with TALP did not proliferate at all. Accordingly, it was confirmed that TALP can be used as anticancer agent, since TALP inhibits cell proliferation like taxol.

### EXAMPLE 10

### Antiviral Activity of TALP

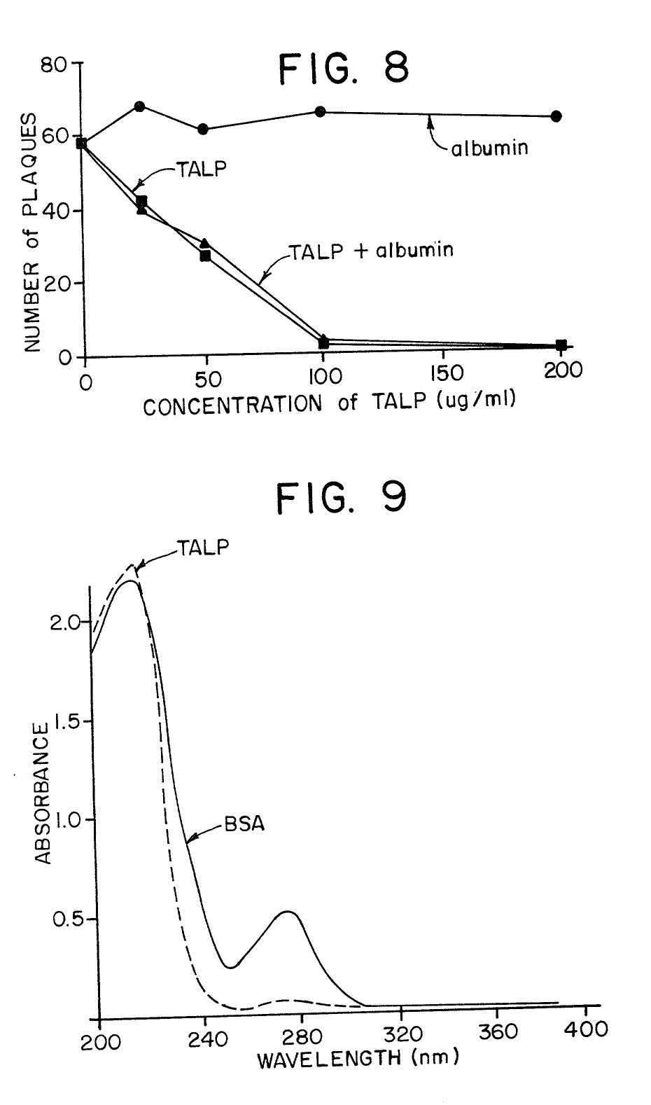

To investigate inhibitory effect of viral proliferation by TALP, albumin filtered with 0.22 μm acrodisc, TALP or mixture of albumin and TALP was added to P4 phage containing solution, and Luria medium(1 g of trypton, 0.5 g of yeast extract and 1 g of NaCl in 100 ml of distilled water, pH 7.5) was added to a final volume of 100 μl. The reaction mixture was incubated for 30 minutes at 37° C., and E. coli C117 was added and mixed well. Then, 4 ml of Luria medium containing 0.7% agarose was added to the mixture, and the mixture was poured into the plate where 6 ml of Luria medium containing 1.4% agarose was solidified previously. Then, the cells were cultured overnight at 37° C., and number of plaques thus lysated was counted(see: FIG. 8). As shown in FIG. 8, 58 plaques on an average appeared in control plate with albumin only; 30% and 52% of plaque reduction occurred in the plate with 25 μg/ml and 50 μg/ml of TALP, respectively, and almost none of plaques were observed in the plate with 100 μg/ml of TALP; the plate with the mixture of albumin and TALP showed a similar pattern as the plate with TALP only.

Accordingly, it was demonstrated that TALP of the invention can be also used as antiviral agent, since it has antiviral activity.

### EXAMPLE 11

### Wavelength Scanning of TALP

To investigate properties of TALP, changes in absorbance of TALP and BSA were measured employing ultraviolet ray ranging from 200 nm to 340 nm(see; FIG. 9). In FIG. 9, a solid line and a dotted line indicate changes in absorbance of BSA and TALP, respectively. As can be seen in FIG. 9, it was found that TALP had a relatively low value of absorbance at 280 nm compared with BSA, which suggests that amino acid composition of TALP is different from other proteins.

### EXAMPLE 12

### Binding of TALP with αβ-Tubulin and Subtilisin-Digested Tubulin

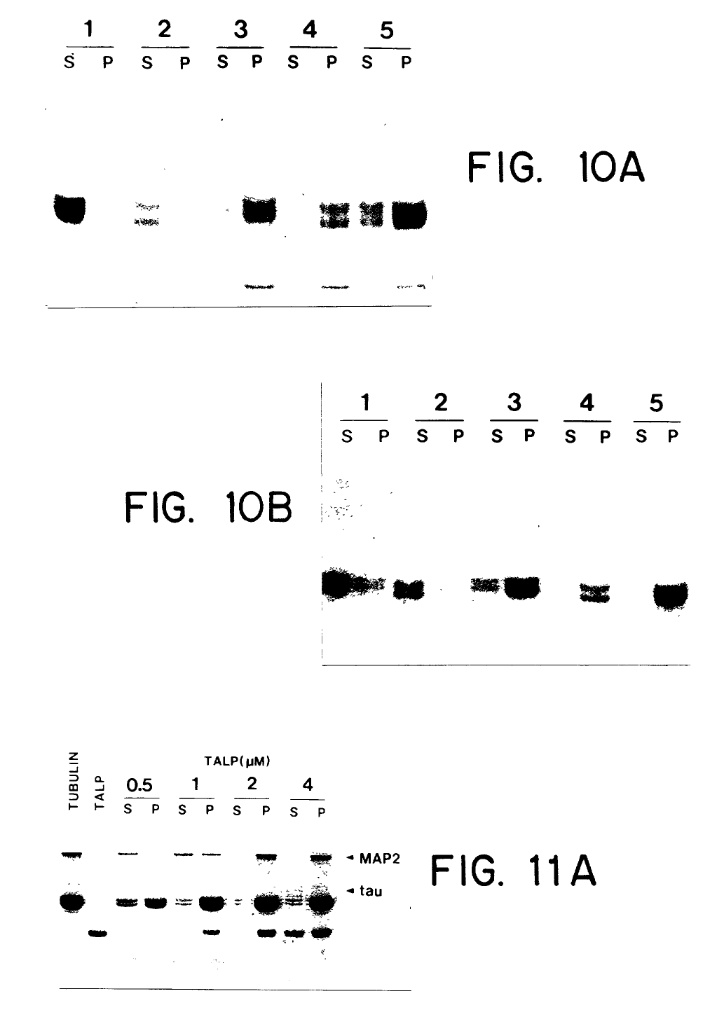

To investigate whether TALP binds to carboxyl-terminus of tubulin molecule, αβ_{S}-tubulin and α_{S}β_{S}-tubulin were prepared, TALP was added to the tubulin for microtubule assembly, and coprecipitation assay was carried out. MAPs without tubulin which were isolated by phosphocellulose chromatography were added to 50 mM MES buffer(pH 6.8) to a concentration of 0.4 mg/ml, and taxol was added to a final concentration of 20 μM, and the reaction mixture was incubated at 37° C. for 30 minutes. Then, the tubulins were digested with 2%(w/w) subtilisin for 30 minutes and 9 hours to prepare αβ_{S}-tubulin and α_{S}β_{S}-tubulin, respectively, and PMSF was added to stop the proteolytic reaction. And, tubulins digested with subtilisin were polymerized at 37° C. for 30 minutes in the presence or absence of 0.4 μM TALP, and centrifuged for 20 minutes. The supernatants and the pellets thus obtained were analyzed by 9% SDS-PAGE(see: FIGS. 10(A) and 10(B)). In FIGS. 10(A) and 10(B), S and P designate supernatant and pellet, respectively. In FIG. 10(A), lane 1 shows αβ-tubulin polymerization; lane 2 shows αβ_{S}-tubin polymerization; lane 3 shows αβ-tubulin polymerization in the presence of 0.4 μM TALP; lane 4 shows αβ_{S}-tubulin polymerization in the presence of 0.4 μM TALP; and, lane 5 shows αβ-tubulin and αβ_{S}-tubulin polymerization in the presence of 0.4 μM TALP, respectively. In FIG. 10(B), lane 1 shows αβ-tubulin polymerization; lane 2 shows α_{S}β_{S}-tubulin polymerization; lane 3 shows αβ-tubulin polymerization in the presence of 0.4 μM TALP; lane 4 shows α_{S}β_{S}-tubulin polymerization in the presence of 0.4 μM TALP; and, lane 5 shows αβ-tubulin and α_{S}β_{S}-tubulin polymerization in the presence of 0.4 μM TALP, respectively.

As shown in FIGS. 10(A) and 10(B), it was found that TALP induced the polymerization of αβ-tubulins and tubulins digested with subtilisin. When TALP was added to the tubulin solution containing αβ-tubulin, αβ_{S}-tubulin or α_{S}β_{S}-tubulin in order to polymerize tubulins, TALP was precipitated together with αβ-tubulin. This result indicates that binding affinity of TALP to intact tubulin is more potent than one to subtilisin-digested tubulins, and high affinity binding site of TALP is located at the carboxyl-terminal domain of tubulin molecule.

### EXAMPLE 13

### Competition of Tau Protein and TALP in Tubulin Polymerization

It has been well known that microtublin assembly-activating tau protein and MAPs bind to C-terminal of tubulin molecules. As can be seen in FIGS. 10(A) and 10(B), TALP also binds to C-terminal of tubulin molecules. In this regard, to determine binding mode of tau protein and MAPs to tubulin in the coexistence of TALP, microtublin assembly employing MAPs-containing tubulin and coprecipitation were carried out in a serial manner.

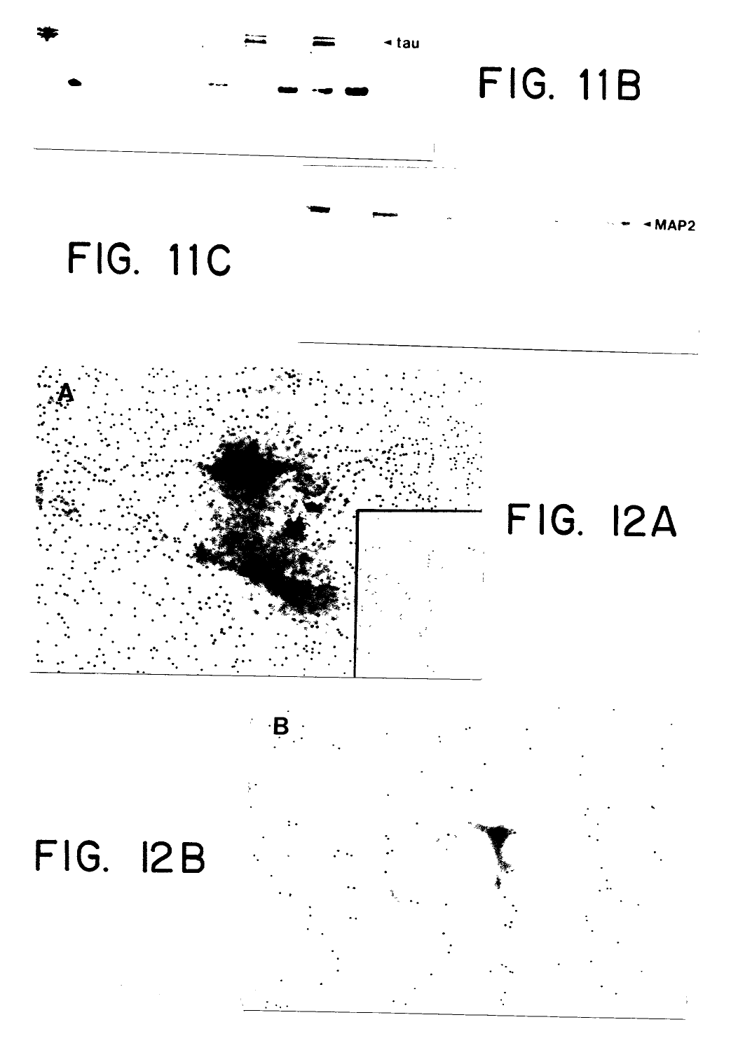

Tubulin which was obtained from bovine brain, was suspended in 50 mM MES buffer(pH 6.8) to the final concentration of 0.4 mg/ml and tubulin was polymerized at 37° C. for 30 minutes after the addition of 0.5, 1, 2 or 4 μM TALP. Centrifugation for 20 minutes followed, and pellet thus obtained was suspended and loaded on 12% SDS-PAGE gel(see: FIG. 11(A)). The gel of FIG. 11(A) was transferred on nitrocellulose membrane and immuno-stained for detection of tau protein and MAP2(see: FIGS. 11(B) and 11(C)). In FIGS. 11(A), 11(B), and 11(C), S and P designate supernatant and pellet, respectively.

As shown in FIG. 11(B), in coexistence of TALP, level of the tau protein bound to tubulin decreased linearly with the increase of TALP concentration, and in the presence of high concentration of TALP, tau protein did not bind at all and TALP bound to tubulin instead of tau protein. However, as shown in FIG. 11(C), in coexistence of TALP, level of the MAP2 bound to tubulin increased linearly with the increase of TALP concentration.

Accordingly, it was confirmed that TALP binds to C-terminal of tubulin molecule, and TALP and tau protein bind competitively to an identical C-terminal of tubulin.

### EXAMPLE 14

### Electron Microscopic Examination of Competitive Binding of Tau Protein and TALP

To examine competitive binding of TALP and tau protein to C-terminal of tubulin, immunogold labelling for tau protein was carried out and examined with scanning electron microscope(Hitachi H-600, JAPAN) at an accelerated voltage of 75 kv. To the reaction mixture containing tubulin and MAPs, were added 2 mM GTP and 4 μM TALP, respectively. Then, polymerization was followed at 37° C. for 30 minutes, crosslinked with 3×0.96% glutaraldehyde at 26° C., dropped on nickel/Formvar-coated grid, and dried. Immunogold labelling was employed by mouse anti-tau protein and anti-mouse IgE conjugated with 12 mm-gold particles, and stained with 1% uranyl acetate for 5 minutes and examined with electron microscope after washing with distilled water and drying(see: FIGS. 12 (A) and 12(B)).

FIGS. 12(A) and 12(B) are 30,000-fold magnified electron micrographs of tau protein immunogold labelling in the presence of 2 mM GTP and 4 μM TALP, respectively. Insert of FIG. 12(A) show an 8,000-fold magnified electron micrograph. As can be seen in FIGS. 12(A) and 12(B), it was demonstrated that gold particle number on microtubulin stabilized by TALP was significantly decreased comparing that of normal microtubulin, which commensurates with the result of immuno-staining for tau protein described in Example 13.

### EXAMPLE 15

### Inhibition of Proliferation of Human Large Intestine Cancer HM7 Cell Line

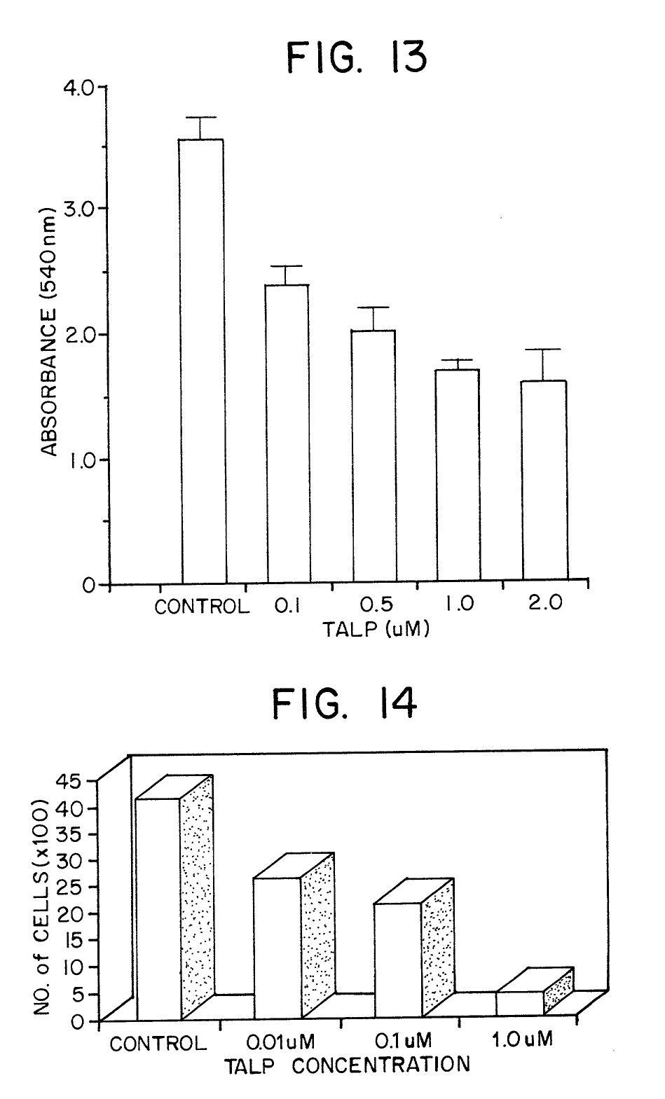

To determine inhibitory effects of TALP on the multiplicity of human cancer cell, HM7 cell line which is derived from human colon cancer LSI74T(see: Kuan S. F. et al., Cancer Res., 47:5715-5724(1987); Kuan S. F. et al., J. Biol. Chem., 264:19271-19277(1989)) was coated at a concentration of 10⁴ cells/200 μl on 96-well microplates and post 4-day incubation, TALP was added at a different concentration of 0.1, 0.5, 1 and 2 μM whose concentration was selected from the experimental data that TALP in a concentration of 2 μM or below revealed no cytotoxicity on HM7 cell. Incubation for 4 days thereafter followed and cell number was calculated by measuring absorbance at 540 nm in accordance with the MTT method(see: FIG. 13).

As shown in FIG. 13, inhibition rate(%) of HM7 cell multiplicity alas determined to be 33.3%, 43.8%, 52.23% and 55.14%, respectively, depending on the treated TALP concentration, and ID₅₀ was assumed to be 0.83 μM. Accordingly, it was demonstrated that TALP can repress proliferation of human cancer cell, even at a low concentration.

### EXAMPLE 16

### In Vitro Motility Assay

If stimulation and stabilization of microtubule assembly in HM7 cell line by TALP leads to inhibition of cell proliferation, motility of HM7 cell could be expected naturally to decrease. Therefore, to verify the expectation, in vitro motility assay was carried out(see: Yoon et al., Biochem. Biophys. Res. Commun., 222:694-699(1996)) by employing Transwell cell culture chamber equipped with 6.5 mm polycarbonate filter in diameter whose pore size is 8 μM as follows.

200 μl of HM7 cell suspension(2×10⁵ cells/200 μl) was added to upper compartment of the Transwell chamber, and 800 μl of cell culture media was added to lower compartment together with 0.01, 0.1 and 1 μM of TALP. After incubation for 3 days, cells adhered to upper region of filter were removed by cotton-wool and the filter was stained with haematoxylin to determine cell number transfered to the lower region of filter under a microscope with magnifying power of 100 (see: FIG. 14). As can be seen in FIG. 14, cell number of control group was determined to be 41.33±3.32, and the experimental groups treated with 0.01 μM, 0.1 μM and 1 μM TALP were 26.13±6.05, 21.33±2.5 and 4.44±2.07, respectively, which corresponds to 36.8%, 48.4% and 89.3% inhibition of cell motility.

Therefore, it was clearly determined that TALP inhibits cell motility in an effective manner even under a low concentration of 1 μM below. This hints TALP could be used as an anti-metastasis agent, since in vitro cell motility assay shows both cell multiplicity and motility of the tested cell.

### EXAMPLE 17

### In Vivo Inhibitory Test of Metastasis

To confirm whether inhibitory effects of cell proliferation and motility by TALP described in Examples 15 and 16 result in a significant decrease of metastasis, in vivo test was carried out by employing nude mouse.

HM7 cell cultured in 75 cm² tissue culture flask was treated with trypsin for several minutes, washed with PBS several times and suspended in serum-free media to a concentration of 10⁷ cells/ml. 100 μl of the suspension was injected into spleen of BALB/C/nu/nu athymic nude mouse(female) of 4 weeks and the spleen was cut out lapsing 1 minute. Then, 100 μl of TALP(50 μg/100 μl) was injected intraperitoneally once a day during 8 days, i.e., from the removal of spleen to 7 days thereafter. The mouse was sacrificed after 4 weeks, and weight of liver and number of the foci where metastasis into liver occurred were determined. As a control, 100 μl of sterile saline was injected instead of TALP.

Liver weight of control group was determined to be 5.05 g in average which corresponds to 3.7 times of TALP-treated group of 1.388 g in average, and the number of foci was determined to be control group and 73 in case of TALP-treated group, which means 82.8% decrease of foci number. Accordingly, it was determined that TALP plays dual role as anticancer and anti-metastasis agents.

As clearly illustrated and demonstrated as aboves, the present invention provides a novel taxol-like protein (TALP) , process for preparing the same, and use of the same as anticancer and antiviral agents. TALP of the invention promotes tubulin polymerization without GTP, like taxol, and the microtubules thus polymerized are resistant to disassembly caused by Ca²⁺ and cold temperature under 4° C. and highly stabilized. Moreover, TALP of the invention inhibits cell proliferation, regeneration of scraped NIH 3T3 cell, proliferation and in vitro motility of HM7 human colon cancer cell, cell division of fertilized Xenopus oocyte, and E. coli lysis caused by virus.

What is claimed is:

1. A taxol-like protein(TALP) having a molecular weight of about 35 kDa, which is isolated from mammalian tissue and capable of promoting microtubule assembly.

2. The taxol-like protein(TALP) of claim 1, wherein the mammalian tissue is human placenta.

3. The taxol-like protein(TALP) of claim 1, wherein the microtubule assembled by TALP is resistant to disassembly caused by Ca²⁺ and cold temperature under 4° C.

4. The taxol-like protein(TALP) of claim 1, wherein the microtubule assembly is promoted without GTP or microtubule-associated proteins.

5. The taxol-like protein(TALP) of claim 1, which inhibits cell proliferation.

6. The taxol-like protein(TALP) of claim 1, which inhibits viral proliferation.

7. The taxol-like protein(TALP) of claim 1, which inhibits cellular motility.

8. The taxol-like protein(TALP) of claim 1, which inhibits metastasis of cancer cell.

9. The taxol-like protein(TALP) of claim 1, which inhibits binding of tau protein to tubulin molecule.

10. A process for preparing the taxol-like protein(TALP) of claim 1 which comprises the steps of:
(i) homogenizing mammalian tissue and centrifuging to obtain supernatant; and,
(ii) fractionating the supernatant thus obtained by cation-exchange chromatography and hydroxyapatite chromatography.
